(19) United States
(12) Patent Application Publication
(10) Pub. No.: US 2005/0124581 A1
(43) Pub. Date: Jun. 9, 2005

(54) Alpha-substituted heteroarylalkyl phosphonate derivatives

(76) Inventors: Hieu Trung Phan, Tannay (CH); Lan Mong Nguyen, Nyon (CH); Raymond Azoulay, Geneve (CH); Vinh Van Diep, Vetraz-Monthoux (FR); Eric Joseph Niesor, Nyon (CH); Craig Leigh Bentzen, Bogis-Bossey (CH); Robert John Ife, Stevenage, Hertfordshire (GB)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/504,081

(22) PCT Filed: Feb. 3, 2003
(86) PCT No.: PCT/US03/03091

Related U.S. Application Data
(60) Provisional application No. 60/355,864, filed on Feb. 11, 2002.

(51) Int. Cl. A61K 31/675

(52) U.S. Cl. 514/79; 514/89; 514/85; 544/243; 544/232; 546/22; 548/112

(57) ABSTRACT

The present invention relates to novel α-substituted heteroarylalkylphosphonate derivatives and their uses for lowering plasma levels of apo (a), Lp(a), apo B, apo B associated lipoproteins (low density lipoproteins and very low density lipoproteins) and for lowering plasma levels of total cholesterol.

### FIELD OF THE INVENTION

This invention relates to substituted heteroarylalkylphosphonate compositions and therapeutic uses thereof. More specifically, the present invention relates to novel α-substituted heteroarylalkylphosphonate derivatives, processes for their preparation, pharmaceutical compositions containing them and their use in therapy for lowering plasma levels of apo (a) and apo (a) associated lipoprotein (lipoprotein(a) or “Lp(a)”), for lowering plasma levels of apo B and apo B associated lipoproteins (low density lipoproteins and very low density lipoproteins), and for lowering plasma levels of total cholesterol.

### BACKGROUND OF THE INVENTION

Lp(a) is a LDL-like lipoprotein wherein the major lipoprotein, apo B-100, is covalently linked to an unusual glycoprotein, apoprotein(a). The covalent association between apo(a) and apo B to form Lp(a) is a secondary event which is independent of the plasma concentration of apo B. Due to its structural similarity to plasminogen, apo(a) interferes with the normal physiological thrombosis-hemostasis process by preventing thrombolysis, that is clot dissolution (see e.g., Biemond B J, Circulation 1997, 96(5) 1612-1615). The structural feature of Lp(a), where the LDL lipoprotein is linked to apo(a), is thought to be responsible for its atherogenic and thrombogenic activities.

Elevated levels of Lp(a) have been associated with the development of atherosclerosis, coronary heart disease, myocardial infarction, cerebral infarction, restenosis following balloon angioplasty and stroke. A recent epidemiologic study has provided the clinical proof of a positive correlation between plasma Lp(a) concentrations and the incidence of heart disease (A. G. Bostorn, et al., Journal of American Medical Association 1996, 276, p. 544-548).

Patients that have Lp(a) levels in excess of 20-30 mg/dl run a significantly increased risk of heart attacks and stroke. An effective therapy for lowering Lp(a) does not exist at present because cholesterol lowering agents such as the HMGCoA reductase inhibitors do not lower Lp(a) plasma concentrations. The only compound that lowers Lp(a) is niacin, but the high doses necessary for activity are accompanied with unacceptable side-effects. There is, therefore, an unmet therapeutic need for agents that effectively reduce elevated levels of Lp(a).

International applications WO 97/20307, WO 98/28310, WO 98/28311 and WO 98/28312 (Symphar, SmithKline Beecham) describe a series of α-amino phosphonates which have Lp(a) lowering activity. There however remains the need to identify further compounds having Lp(a) lowering activity.

### SUMMARY OF THE INVENTION

The present invention provides, in a first aspect, a compound of formula (Ia): 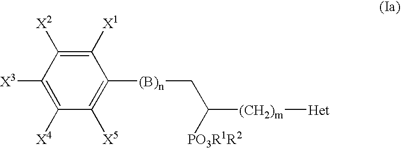
or a compound of formula (Ib): 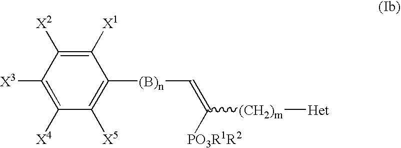
in which X¹, X², X³, X⁴ and X⁵ are independently hydrogen, hydroxy, hydroxymethyl, C₁-C₃ alkoxymethyl, straight or branched C₁-C₈ alkyl, straight or branched C₁-C₈ alkoxy, C₃-C₆ cycloalkyl, C₃-C₆ cycloalkoxy, cyano, nitro or halogen, wherein said halogen is fluoro, chloro, bromo or iodo; or X² may be combined with X³, or X⁴ may be combined with X⁵, to form a 5- to 6-membered alkylidenedioxy ring optionally substituted with a C₁-C₄ alkyl group; X⁴ may be combined with X⁵ to form a 5- to 6-membered alkylidene ring optionally substituted with a C₁-C₄ alkyl group;
R¹ and R² are independently hydrogen or a straight or branched C₁-C₆ alkyl;
B is CH₂, CH₂—CH₂, CH═CH;
n is zero or 1;
m is zero, 1 or 2;
Het is an optionally substituted heteroaryl group comprising at least one nitrogen atom, or a pharmaceutically acceptable salt thereof.

The compound of formula (Ib) may be the Z-isomer, formula (Ib^{Z}): 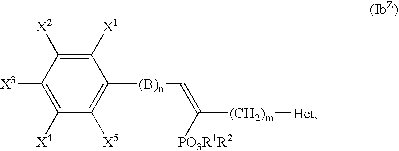
or the E-isomer, formula (Ib^{E}): 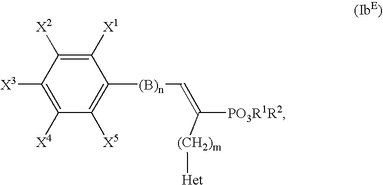
or a mixture thereof.

Compounds of the present invention include:
(E)-diethyl β-(3-ethoxy-4-hydroxyphenyl)-α-(3-pyridyl)vinylphosphonate;
diethyl β-(3-ethoxy-4-hydroxyphenyl)-α-(3-pyridyl)ethylphosphonate;
(E)-diethyl β-(4-hydroxy-2,3,5-trimethylphenyl)-α-(3-pyridyl)vinylphosphonate;
diethyl β-(4-hydroxy-2,3,5-trimethylphenyl)-α-(3-pyridyl)ethylphosphonate;
(E)-diethyl β-(3,5-dimethoxy-4-hydroxyphenyl)-α-(3-pyridyl)vinylphosphonate;
diethyl β-(3,5-dimethoxy-4-hydroxyphenyl)-α-(3-pyridyl)ethylphosphonate;
(E)-diethyl β-(3,5-dimethoxy-4-hydroxyphenyl)-α-(5-(2-methylpyridyl))vinylphosphonate;
diethyl β-(3,5-dimethoxy-4-hydroxyphenyl)-α-(5-(2-methylpyridyl))ethylphosphonate;
diisopropyl β-(3,5-dimethoxy-4-hydroxyphenyl)-α-(5-(2-methylpyridyl))ethylphosphonate;
(E)-diethyl β-(4-hydroxy-3-methoxy-5-methylphenyl)-α-(3-pyridyl)vinylphosphonate;
diethyl β-(4-hydroxy-3-methoxy-5-methylphenyl)-α-(3-pyridyl)ethylphosphonate;
(E)-diethyl β-(4-hydroxy-3-methoxy-5-methylphenyl)-α-(5-(2-methylpyridyl)) vinylphosphonate;
diethyl β-(4-hydroxy-3-methoxy-5-methylphenyl)-α-(5-(2-methylpyridyl)) ethylphosphonate;
diisopropyl β-(4-hydroxy-3-methoxy-5-methylphenyl)-α-(5-(2-methylpyridyl)) ethylphosphonate;
(E)-diethyl β-(3,5-dimethoxy-4-hydroxyphenyl)-α-(4-(2-methylthiazolyl))vinylphosphonate;
(E)-diethyl β-(4-hydroxy-3-methoxy-5-methylphenyl)-α-(4-(2-methylthiazolyl)) vinylphosphonate;
(E)-diethyl β-(4-hydroxy-3-methoxy-5-methylphenyl)-α-(pyrazinyl)vinyl phosphonate; and
diethyl β-(4-hydroxy-3-methoxy-5-methylphenyl)-α-(pyrazinyl)ethylphosphonate.

One aspect of the present invention provides for a pharmaceutical composition comprising a compound of formula (Ia) or formula (Ib) and a pharmaceutically acceptable excipient. Hereinafter compounds of formula (Ia) and compounds of formula (Ib) are collectively termed “compounds of formula (I).”

The present invention also provides for therapeutic uses of the compounds of formula (I). In one aspect, the invention provides for a method of decreasing plasma levels of apo (a) and lipoprotein(a), in reducing plasma levels of apo B and LDL cholesterol and in decreasing plasma total cholesterol. The present invention also provides further methods including: a method of prevention and/or treatment of thrombosis by increasing thrombolysis through decreasing plasma levels of apo (a) and lipoprotein(a); a method of treatment of restenosis following angioplasty by decreasing plasma levels of apo (a) and lipoprotein(a); a method of prevention and/or treatment of atherosclerosis by decreasing plasma levels of apo (a) and lipoprotein(a) or by decreasing plasma levels of apoprotein B and LDL cholesterol; a method of prevention and/or treatment of hypercholesterolemia; a method of prevention and/or treatment of atherosclerosis by lowering cholesterol in patients that are resistant to treatment with statins; and a method of prevention and/or treatment of atherosclerosis in association with a compound such as a statin which decreases cholesterol synthesis.

### DETAILED DESCRIPTION OF THE INVENTION

The present invention relates to the compounds of formula (I) and their uses for lowering plasma levels of apo (a), Lp(a), apo B, apo B associated lipoproteins (low density lipoproteins and very low density lipoproteins) and for lowering plasma levels of total cholesterol.

In relation to compounds of formula (I), in preferred embodiments, X¹ is hydrogen, or methyl, X² is methoxy, ethoxy, methyl, tert-butyl or hydroxy, X³ is hydrogen, hydroxy, methoxy, methyl, ethyl or hydroxymethyl, X⁴ is hydrogen, methoxy, methyl or tert-butyl and X⁵ is hydrogen. In a preferred combination, X² is methoxy, X³ is hydroxy and X⁴ is methyl or methoxy. Preferably, n is zero, so that (B)ₙ is replaced with a direct bond. Preferably R¹ and R² are C₁-C₃ alkyl, more preferably C₂ or C₃, and in particular wherein R¹ and R² are independently ethyl or isopropyl. Preferably m is zero or 1.

When used herein the term “heteroaryl” refers to, unless otherwise defined, a single or a fused ring containing up to four heteroatoms in each ring, each of which is selected from oxygen, nitrogen and sulphur, which rings may be unsubstituted or substituted by, for example, up to four substituents. Each ring suitably has from 4 to 7, preferably 5 or 6 ring atoms. A fused ring system may include carbocyclic rings and need include only one heteroaryl ring.

Representative examples of Het include pyridyl, pyrimidyl, pyrazinyl, pyridazinyl, thiazolyl, thiadiazolyl, benzothiazolyl, isoxazolyl, pyrazolyl, triazinyl, and imidazolyl which may be unsubstituted or substituted by up to four substituents (for pyridyl and benzothiazolyl), three substituents (pyrimidyl, pyrazinyl, pyridazinyl, pyrazolyl), two substituents (thiazolyl, isoxazolyl, triazinyl and imidazolyl) or one substituent (thiadiazolyl) which may be the same or different and selected from straight or branched C₁-C₄ alkyl or alkoxy, hydroxy, hydroxymethyl, halogen (F, Cl, Br, I), or an amino group optionally substituted with C₁-C₄ alkyl. Preferably, pyridyl, pyrimidyl, pyrazinyl, pyridazinyl, thiazolyl, thiadiazolyl, benzothiazolyl, pyrazolyl, or triazinyl is unsubstituted or substituted by methyl, methoxy, dimethoxy or dimethyl. Preferred examples of Het include is pyrazinyl, 3-pyridyl, 5-(2-methylpyridyl), 5-(2-methylthiazolyl) pyridyl).

Pharmaceutically acceptable salts for use in the present invention include those described by Berge, Bighley, and Monkhouse, J. Pharm. Sci., 1977, 66, 1-19. Such salts may be formed from inorganic and organic acids. Representative examples thereof include maleic, fumaric, benzoic, ascorbic, parnoic, succinic, bismethylenesalicylic, methanesulfonic, ethanedisulfonic, acetic, propionic, tartaric, salicylic, citric, gluconic, aspartic, stearic, palmitic, itaconic, glycolic, p-aminobenzoic, glutamic, benzenesulfonic, hydrochloric, hydrobromic, sulfuric, cyclohexylsulfamic, phosphoric and nitric acids.

It will be appreciated that certain compounds of the present invention, in particular those of formula (Ia), will comprise one or more chiral centres so that compounds may exist as stereoisomers, including diastereoisomers and enantiomers. The present invention covers all such stereoisomers, and mixtures thereof, including racemates. The compounds of formula (Ib) of the present invention comprise the individual E- and Z-diastereoisomers and mixtures thereof.

Since the compounds of the present invention are intended for use in pharmaceutical compositions, it will be understood that they are each provided in substantially pure form, for example at least 50% pure, more suitably at least 75% pure and preferably at least 95% pure (% are on a wt/wt basis). Impure preparations of the compounds of formula (I) may be used for preparing the more pure forms used in the pharmaceutical compositions. Although the purity of intermediate compounds of the present invention is less critical, it will be readily understood that the substantially pure form is preferred as for the compounds of formula (I). Preferably, whenever possible, the compounds of the present invention are obtained in crystalline form.

When some of the compounds of this invention are allowed to crystallise or are recrystallised from organic solvents, solvent of crystallisation may be present in the crystalline product. This invention includes within its scope such solvates. Similarly, some of the compounds of this invention may be crystallised or recrystallised from solvents containing water. In such cases water of hydration may be formed. This invention includes within its scope stoichiometric hydrates as well as compounds containing variable amounts of water that may be produced by processes such as lyophilisation. In addition, different crystallisation conditions may lead to the formation of different polymorphic forms of crystalline products. This invention includes within its scope all polymorphic forms of the compounds of formula (I).

The present invention also relates to the unexpected discovery that compounds of formula (I) are effective for decreasing apo(a) production in vitro and Lp(a) production in vivo in Cynomolgus monkeys. This species has been selected as the animal model as its Lp(a) is similar in immunologic properties to human Lp(a) and occurs in almost identical frequency distribution of plasma concentrations, see e.g., N. Azrolan et al; J. Biol. Chem., 266, 13866-13872 (1991). In the ill vitro assay, compounds of formula (I) have been shown to reduce the secretion of apo (a) which is secreted in free form from the primary cultures of the Cynomolgus monkey hepatocytes. These results are confirmed by the in vivo studies performed on the same animal species showing the potent decrease of Lp(a) by compounds of formula (I). Therefore the compounds of this invention are useful for decreasing apo (a) and Lp(a) in man and thus provide a therapeutic benefit.

Accordingly in a further aspect, this invention provides a compound of formula (I) or a pharmaceutically acceptable salt thereof for use in therapy, in particular as a Lp(a) lowering agent. Elevated plasma and tissue levels of Lp(a) are associated with accelerated atherosclerosis, abnormal proliferation of smooth muscle cells and increased thrombogenesis and expressed in disease states such as, for instance: coronary heart disease, peripheral artery disease, intermittent claudication, thrombosis, restenosis after angioplasty, extra-cranial carotid atherosclerosis, stroke and atherosclerosis occurring after heart transplantion.

Furthermore, the compounds of the present invention may possess cholesterol lowering properties and decrease total plasma cholesterol, in particular LDL cholesterol. It is now well established that a high level of LDL cholesterol is a major risk factor for atherosclerotic diseases. In addition, the compounds of the present invention may decrease the levels of apoprotein B (apo B) which is the main protein of LDL and the main ligand for LDL receptors. The mechanism of decrease in apo B and in apo B-associated LDL probably does not involve inhibition of cholesterol synthesis, which is the mechanism demonstrated for the statins. Therefore, compounds of the present invention are useful for lowering cholesterol in patients who are resistant to treatment with a statin, and, conversely, also have an additive or synergistic effect for lowering cholesterol in those patients who are responding to treatment with statins.

Thus, compounds of the present invention are of use in therapy as cholesterol lowering agents. Furthermore, a dual profile in lowering plasma Lp(a) and plasma cholesterol makes the compounds of formula (I) useful in therapy for the prevention and/or treatment of both the acute and chronic aspects of atherosclerosis.

Compounds of the present invention may also be of use in preventing and/or treating the above-mentioned disease states in combination with anti-hyperlipidaemic, anti-atherosclerotic, anti-diabetic, anti-anginal, anti-inflammatory or anti-hypertension agents. Examples of the above include cholesterol synthesis inhibitors such as statins, for instance atorvastatin, simvastatin, pravastatin, cerivastatin, fluvastatin, lovastatin and ZD 4522 (also referred to as S-4522′, Astra Zeneca), anti-oxidants such as probucol, insulin sensitisers such as a PPAR gamma activator, for instance G1262570 (Glaxo Wellcome) and the glitazone class of compounds such as rosiglitazone (Avandia, SmithKline Beecham), troglitazone and pioglitazone, calcium channel antagonists, and anti-inflammatory drugs such as NSAIDs.

For therapeutic use the compounds of the present invention will generally be administered in a standard pharmaceutical composition. Accordingly in a further aspect, the invention provides for a pharmaceutical composition comprising a compound of formula (I) or a pharmaceutically acceptable salt thereof and a pharmaceutically acceptable excipient or carrier. Suitable excipients and carriers are well known in the art and will be selected with regard to the intended route of administration and standard pharmaceutical practice. For example, the compositions may be administered orally in the form of tablets containing such excipients as starch or lactose, or in capsules, ovules or lozenges either alone or in admixture with excipients, or in the form of elixirs or suspensions containing flavoring or coloring agents. They may be injected parenterally, for example, intravenously, intramuscularly or subcutaneously. For parenteral administration, they are best used in the form of a sterile aqueous solution which may contain other substances, for example, enough salts or glucose to make the solution isotonic with blood. The choice of form for administration as well as effective dosages will vary depending, inter alia, on the condition being treated. The choice of mode of administration and dosage is within the skill of the art.

The compounds of formula (I) and their pharmaceutically acceptable salts which are active when given orally can be formulated as liquids, for example syrups, suspensions or emulsions or as solids for example, tablets, capsules and lozenges. A liquid formulation will generally consist of a suspension or solution of the compound or pharmaceutically acceptable salt in suitable liquid carrier(s) for example, ethanol, glycerine, non-aqueous solvent, for example polyethylene glycol, oils, or water with a suspending agent, preservative, flavoring or coloring agents. A composition in the form of a tablet can be prepared using any suitable pharmaceutical carrier(s) routinely used for preparing solid formulations. Examples of such carriers include magnesium stearate, starch, lactose, sucrose and cellulose. A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

Typical parenteral compositions consist of a solution or suspension of the compound or pharmaceutically acceptable salt in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration. A typical suppository formulation comprises a compound of structure (I) or a pharmaceutically acceptable salt thereof which is active when administered in this way, with a binding and/or lubricating agent such as polymeric glycols, gelatins or cocoa butter or other low melting vegetable or synthetic waxes or fats. Preferably the composition is in unit dose form such as a tablet or capsule.

Each dosage unit for oral administration contains preferably from 1 to 250 mg (and for parenteral administration contains preferably from 0.1 to 25 mg) of a compound of formula (I) or a pharmaceutically acceptable salt thereof calculated as the free base.

The compounds of the invention will normally be administered to a subject in a daily dosage regimen. For an adult patient this may be, for example, an oral dose of between 1 mg and 500 mg, preferably between 1 mg and 250 mg, or an intravenous, subcutaneous, or intramuscular dose of between 0.1 mg and 100 mg, preferably between 0.1 mg and 25 mg, of the compound of the formula (I) or a pharmaceutically acceptable salt thereof calculated as the free base, the compound being administered 1 to 4 times per day.

The present invention also relates to processes for preparing novel α-substituted heteroarylalkylphosphonate derivatives of formula (I), which is described below.

Compounds of formula (Ib) may be prepared by a process which comprises condensing an aldehyde of formula (II): 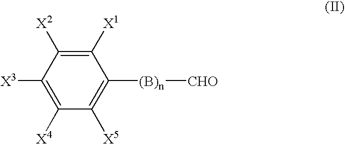
in which X¹, X², X³, X¹, X⁵, B and n are as previously defined; with an heteroarylalkylphosphonate of formula (III): 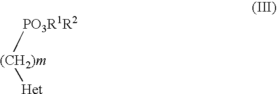
in which m, R¹, R² and Het are as previously defined,

The condensation reaction between (II) and (III) can be carried out in several ways. In the first variant the α-silyl carbanion of the heteroarylalkyl phosphonate (III) is condensed with the aldehyde (II) under the conditions of the Peterson olefination reaction. Suitable silylating reagents include chlorotrimethylsilane or chlorotriethylsilane. A preferred silylating agent is chlorotrimethylsilane. Suitably, the condensation may be carried out in an ether solvent such as diethyl ether, tetrahydrofuran (TBF), dimethoxyethane or dioxane. A preferred solvent is THF. Suitable bases include n-butyllithium, lithium diisopropylamide (LDA) formed in situ by reacting n-butyllithium and diisopropylamine, or n-butyllithium used in association with N,N,N′,N′-tetramethylethylenediamine The reaction is suitably carried out in the range from −78° C. to room temperature (20° C.).

Another variant consists in reacting the carbanion of the heteroarylalkyldiphosphonate (IV): 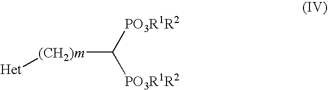
with the aldehyde (II) under the Horner-Emmons olefination reaction. Suitably, the condensation may be carried out in an ether solvent such as diethyl ether, tetrahydrofuran (THF), dimethoxyethane, dioxane, or dimethylformamide (DMF). A preferred solvent is THF. Suitable bases include sodium hydride, n-butyllithium, lithium diisopropylamide (LDA) formed in situ by reacting n-butyllithium and diisopropylamine, or n-butyllithium used in association with N,N,N′,N′-tetramethylethylenediamine. The reaction is suitably carried out in the range from −78° C. to room temperature (20° C.).

Both of these two mentioned variants of the condensation of a heteroarylalkylphosphonate of formula (II) or a heteroarylalkyldiphosphonate of formula (IV) with an aldehyde of formula (II) afford compounds of formula (Ib^{Z}) and (Ib^{E}). The two isomers (Ib^{Z}) and (Ib^{E}) can be separated by column chromatography. The structures of these isomers are ascertained by spectroscopic means: MS and in particular NMR, thanks to the characteristic absorption of the olefinic proton. In the (Z)-isomer (Ib^{Z}), the olefinic proton displays a large coupling constant, J=ca 40-43 Hz, due to the trans H—C═C—P coupling. In the (E)-isomer (Ib^{E}) this value is much smaller, J=ca 25 Hz, due to the cis H—C═C—P coupling.

Compounds of formula (Ia) can be prepared by reducing compounds of formula (Ib) or, very conveniently, a mixture of both.

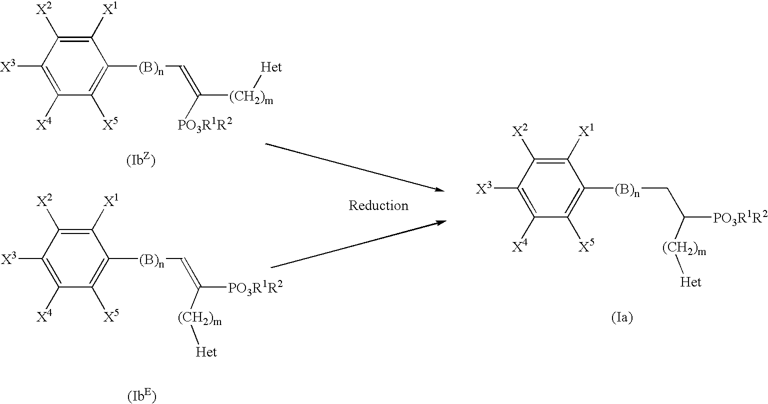

A suitable reduction method is the catalytic hydrogenation using as catalysts palladium or platinum adsorbed on charcoal in a solvent such as ethanol or acetic acid at a pressure between 1 and 4 atm and a temperature between room temperature and 40° C. The reduction can also be carried out by means of a complex hydride reagent such as sodium borohydride or sodium cyanoborohydride in a polar solvent such as methanol, ethanol, isopropanol or n-propanol at a temperature between room and reflux temperature. A further convenient reduction method is the use of a zinc modified sodium cyanoborohydride reagent generated from a mixture of NaBH₃CN:ZnCl₂ in a 2:1 molar ratio in a solvent selected from diethyl ether, tetrahydrofuran, dimethoxyethane and methanol at a temperature between room temperature and reflux temperature; the reaction can be accelerated by the addition of a higher boiling solvent selected from ethanol, isopropanol, n-propanol, isobutanol or n-butanol and heating to reflux the resulting mixture.

In a further variant, compound (Ia) can be directly obtained by the reaction between the heteroarylalkylphosphonate (III) and an alkyl halide of formula (V), wherein the Hal is chloro or bromo, in presence of a base.

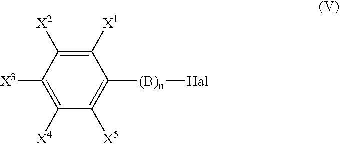

Suitable solvents include diethyl ether, tetrahydrofuran (THF), dimethoxyethane or dioxane. A preferred solvent is THF. Suitable bases include n-butyllithium, lithium diisopropylamide (LDA) formed in situ by reacting n-butyllithium and diisopropylamine, or n-butyllithium used in association with TMEDA (N,N, N′,N′-tetramethylethylenediamine). The reaction is suitably carried out in the range from −78° C. to room temperature (20° C.).

When any of the substituents X¹, X², X³, X⁴ or X⁵, is a hydroxy group, giving a reactive phenol or hydroxymethylphenyl group, it may be useful to protect such a hydroxy group, to avoid troublesome side reactions which may otherwise occur under the strongly alkaline reaction conditions employed. A particularly effective way of protecting the OH group is to convert it into an alkyl silyl ether, such as trimethyl silyl ether (Me₃Si ether or Tms ether) or a t-butyldimethyl silyl ether (tBuMe₂Si ether or Tbs ether). An integral part of this invention is the conversion of the aldehyde of formula (II) or the halide of formula (V) comprising a hydroxy group into the corresponding Tbs ether. Suitable protection reaction conditions are the use of t-butyldimethylsilyl chloride in presence of imidazole in dimethylformamide. Such an Tbs protected aldehyde (II) or halide (V) can then withstand the strongly alkaline conditions which are necessary to form the desired Tbs-protected (Ia) or (Ib) structures. The Tbs protecting group can then be cleaved by fluoride reagents well established in the art to yield the end products of formula (I) wherein any of the substituents X¹, X², X³. X⁴ or X⁵ can be a hydroxy group. Suitable deprotection reaction conditions involve reacting the Tbs protected compound with tetrabutyl ammonium fluoride in glacial acetic acid.

The various starting compounds heteroarylalkylphosphonates (III), heteroarylalkyldiphosphonates (IV), aldehydes (II) and halide (V) can be prepared according to methods described in the chemical literature.

### EXAMPLES OF THE INVENTION

The invention is further described in the following examples that are intended to illustrate the invention without limiting its scope. The abbreviations used in this application are the following: in the tables, n is normal, i is iso, s is secondary and t is tertiary. In the description of the NMR spectra, respectively s is singlet, d doublet, dd double doublet, t triplet, q quadruplet and m multiplet. The temperatures were recorded in degrees Celsius and the melting points are not corrected.

The structures of compounds described in the Examples were established by their infrared (IR), mass (MS) and nuclear magnetic resonance (NMR) spectra. The purity of the compounds was checked by thin layer, gas, liquid or high performance liquid chromatography.

Unless otherwise indicated, the physical constants and biological data given for compounds of formula (Ia) refer to racemates while those given for compounds of formula (Ib^{E}) and (Ib^{Z}) refer to pure isomers.

### Example 1

### (E)-Diethyl β-(4-hydroxy-3-methoxy-5-methylphenyl)-α-(3-pyridyl)-vinyl Phosphonate

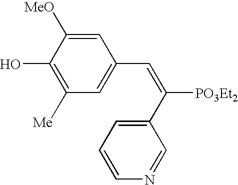

60% NaH (8.63 g, 216 mmol) was washed three times with hexane and was suspended in 60 ml THF. This suspension was cooled to 0° and diethyl phosphite (27.8 ml, 216 mmol) was added dropwise. 30 Minutes after the end of the addition a solution of 3-chloromethylpyridine (13.8 g, 108 mmol) in 60 ml THF was added dropwise and the ice bath was removed. H₂O (40 ml) was added dropwise after stirring at room temperature for 4 h, then sat. NH₄Cl solution (40 ml) was added in one portion. The aqueous phase was separated and extracted with CHCl₃ (3 portions of 200 ml). The combined organic layers were dried with MgSO₄ and evaporated to give 25.8 g of a brown oil. Purification of this crude product by column chromatography (CHCl₃/MeOH 9/1) yielded 20.5 g (89 mmol, 82%) of a brown oil; GC-analysis indicated a purity of 97%.

The whole procedure was carried out at −78° C. and under a nitrogen atmosphere. Diisopropylamine (37.8 ml, 268 mmol) was added dropwise to a solution of nBuLi 1.6 M (168 ml, 268 mmol) in 650 ml THF. After 30 min. a solution of diethyl 3-pyridylmethylphosphonate (20.5 g, 89 mmol) in 50 ml THF was added dropwise. After 30 min of stirring trimethyl chlorosilane (22.5 ml, 178 mmol) was added dropwise, the reaction mixture was stirred for a further 30 min then a solution of 4-t-butyldimethylsilyloxy-3-methoxy-5-methylbenzaldehyde (25 g, 89 mmol) in 50 ml THF was added dropwise. The reaction mixture was stirred at −78° for 2 h, then the cooling bath was removed and sat. NH₄Cl-solution (300 ml) was added in one portion. The mixture was allowed to warm to room temperature and the aqueous phase was separated and extracted with ether (one 800 ml and three 500 ml portions). The combined organic layers were dried with MgSO₄ and evaporated to give 45 g of a brown oil. Purification of this crude product by flash chromatography (AcOEt/MeOH 9/1) yielded 31 g (63 mmol, 70%) of (E)-diethyl β-(4-t-butyldimethylsilyloxy-3-methoxy-5-methylphenyl)-α-(3-pyridyl)-vinylphosphonate as a brown oil.

A solution of tetrabutylammonium fluoride (79.5 g, 252 mmol) in 250 ml TBF was added in four portions to a solution of the preceding compound (31 g, 63 mmol) in 250 ml THF and 67 ml acetic acid. The reaction solution was stirred at room temperature for 3 h and was partitioned between 800 ml CH₂Cl₂ and 200 ml H₂O. The organic phase was separated and washed with three portions of 300 ml sat. NaHCO₃ solution. The organic layer was dried with MgSO₄ and evaporated to give 23.8 g of a brown oil. Purification of this crude product by flash chromatography (AcOEt/MeOH 9/1) yielded 18 g (47.7 mmol, 75%) of the title compound as a white solid, mp=102-103° C.

MS (m/e)=377: M⁺, 239 (100%): M⁺—HPO₃Et₂ NMR (CDCl₃): δ=8.58, 8.51, 7.66 and 7.34 (4m, H each): aromatic H, 3-pyridyl 7.6: (d, 1H, J=24 Hz): (Ph)(CH)C═C(P)-pyridine 6.61 and 6.23 (2m, 1H each): aromatic H, substituted phenyl 5.30 (s, 1H): OH 4.16-4.06 (m, 4H): P—O—CH₂—CH₃ 3.47 (s, 6H): Ph-OCH₃ 2.12 (1s, 3H): Ph-CH₃ 1.29 (2t, J=7 Hz, 6H): P—O—CH₂—CH₃

### Example 2

### Diethyl β-(4-hydroxy-3-methoxy-5-methylphenyl)-α-(3-pyridyl)-ethylphosphonate

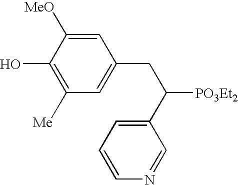

A solution of (E)-diethyl β-(4-hydroxy-3-methoxy-5-methylphenyl)-α-(3-pyridyl)vinylphosphonate (18 g, 47.7 mmol) in 400 ml ethanol was hydrogenated over 9 g of 10% Pd/C catalyst in a Parr hydrogenation apparatus at an initial pressure of 50 psi. When hydrogen uptake has ceased, the catalyst was filtered off, the solvent was evaporated to give 17 g of slightly yellow solid. Purification of this crude product by recrystallisation from a mixture of ligroine and CH₂Cl₂ yielded 13 g (34 mmol, 72%) of a white solid, mp=85-87° C. GC-analysis indicated a purity of 100%.

MS (m/e)=379: M⁺, 241 (100%): M⁺—HPO₃Et₂ NMR (CDCl₃): δ=8.45, 8.38, 7.70 and 7.22 (4m, 1H each): aromatic H, 3-pyridyl 6.39 and 6.21 (2d, 1H each, J=1.5 Hz): aromatic H, substituted phenyl 5.30 (s, 1H): OH 4.11-3.82 (3m, 4H total): P—O—CH₂—CH₃ 3.67 (s, 3H): Ph-OCH₃ 3.44-3.36 (m, H): Ph-CH₂—CH(P)-pyridine 3.3-3.2 and 3.07-2.97 (2m, 1H each): Ph-CH₂—CH(P)-pyridine 2.12 (1s, 3H): Ph-CH₃ 1.30 and 1.15 (2t, J=7 Hz, 3H each): P—O—CH₂—CH₃

### Example 3

### (E)-Diethyl β-(4-hydroxy-3-methoxy-5-methylphenyl)-α-(5-(2-methylpyridyl))-vinylphosphonate

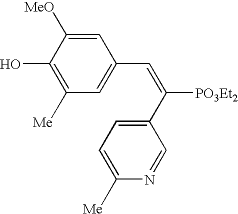

5-Chloromethyl-2-methylpyridine hydrochloride (15 g, 87.3 mmol) was suspended in 100 ml CH₂Cl₂ and a 10% NaOH solution was added while stirring until the pH of the aqueous phase was 8. The mixture was shaken then the CH₂Cl₂ phase was separated, dried over MgSO4 and evaporated to yield 11.9 g (100%) of the free base. 60% NaH (10.63 g, 440 mmol) was washed three times with hexane and was suspended in 100 ml THF. This suspension was cooled to 0° and diethyl phosphite (38.3 ml, 280 mmol) was added dropwise. 30 Minutes after the end of the addition a solution of 5-chloromethyl-2-methylpyridine (17.9 g, 120 mmol) in 10 ml THF was added dropwise and the ice bath was removed. The reaction was stirred for 4 h at room temperature then H₂O (100 ml) was added dropwise, then sat. NH₄Cl solution (100 ml) was added in one portion. The aqueous phase was separated and extracted with CHCl₃ (3 portions of 200 ml). The combined organic layers were dried with MgSO₄ and evaporated to give 25.8 g of a brown oil. Purification of this crude product by column chromatography (CHCl₃/MeOH 95/5) yielded 21.5 g (73%) of diethyl 5-(2-methylpyridyl)methylphosphonate as a brown oil.

The whole procedure was carried out at −78° C. and under a nitrogen atmosphere. Diisopropylamine (2.96 ml, 21 mmol) was added dropwise to a solution of nBuLi 1.6 M (13.2 ml, 21 mmol) in 80 ml THF. After 30 min. a solution of diethyl 5-(2-methylpyridyl)methylphosphonate (1.7 g, 7 mmol) in 100 ml THF was added dropwise. After 30 min of stirring trimethyl chlorosilane (1.77 ml, 14 mmol) was added dropwise, the reaction mixture was stirred for a further 30 min then a solution of 4-t-butyldimethylsilyloxy-3-methoxy-5-methylbenzaldehyde (1.96 g, 7 mmol) in 10 ml THF was added dropwise. The reaction mixture was stirred at −78° for 2 h, then the cooling bath was removed and sat. NH₄Cl-solution (100 ml) was added in one portion. The mixture was allowed to warm to room temperature and the aqueous phase was separated and extracted with ether (one 200 ml and three 100 ml portions). The combined organic layers were dried with MgSO₄ and evaporated to give 2.5 g of a brown oil. Purification of this crude product by column chromatography (AcOEt/MeOH 9/1) yielded 1.3 g (2.5 mmol, 35%) of (E)-diethyl β-(4-t-butyldimethylsilyloxy-3-methoxy-5-methylphenyl)-α-(5-(2-methylpyridyl))-vinylphosphonate as a brown oil.

A solution of tetrabutylammonium fluoride (2.25 g, 7.13 mmol) in 30 ml THF was added in four portions to a solution of the preceding compound (1.3 g, 2.5 mmol) in 20 ml THF and 1.2 ml acetic acid. The reaction solution was stirred at room temperature for 3 h and was partitioned between 500 ml CH₂Cl₂ and 100 ml H₂O. The organic phase was separated and washed with three portions of 300 ml sat. NaHCO₃ solution. The organic layer was dried with MgSO₄ and evaporated to give 2.1 g of a brown oil. Purification of this crude product by flash chromatography (AcOEt/MeOH 9/1) yielded 0.78 g (19.9 mmol, 79%) of the title compound as an oil which slowly crystallized.

MS (m/e)=391: M⁺, 253 (100%): M⁺—HPO₃Et₂ NMR (CDCl₃): δ=8.39, 7.52 and 7.20 (3m, H each): aromatic H, 3-pyridyl 7.58: (d, 1H, J=24 Hz): (Ph)(CH)C═C(P)-pyridine 6.62 and 6.28 (2m, 1H each): aromatic H, substituted phenyl 5.89 (s, 1H): OH 4.16-4.06 (m, 4H): P—O—CH₂—CH₃ 3.50 (s, 6H): Ph-OCH₃ 2.49 (s, 3H): Py-CH₃ 2.12 (1s, 3H): Ph-CH₃ 1.29 (2t, J=7 Hz, 6H): P—O—CH₂—CH₃

### Example 4

### Diethyl β-(4-hydroxy-3-methoxy-5-methylphenyl)-α-(3-pyridyl)-ethylphosphonate

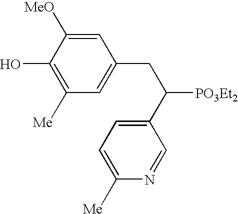

A solution of (E)-diethyl β-(4-hydroxy-3-methoxy-5-methylphenyl)-α-(5-(2-methylpyridyl))vinylphosphonate (0.45 g, 1.15 mmol) in 80 ml ethanol was hydrogenated over 0.2 g of 10% Pd/C catalyst in a Parr hydrogenation apparatus at an initial pressure of 50 psi. When hydrogen uptake has ceased, the catalyst was filtered off, the solvent was evaporated to give 0.6 g of a yellow oil. Purification of this crude product by column chromatography (AcOEt/MeOH 9/1) yielded 0.3 g (0.76 mmol, 66%) of a white solid, mp=68-70° C.

MS (m/e)=393: M⁺, 255 (100%): M⁺—HPO₃Et₂ NMR (CDCl₃): δ=8.35, 7.59 and 7.08 (4m, 1H each): aromatic H, 3-pyridyl 6.39 and 6.22 (2d, 1H each, J=1.5 Hz): aromatic H, substituted phenyl 5.55 (s, 1H): OH 4.11-3.82 (3m, 4H total): P—O—CH₂—CH₃ 3.67 (s, 3H): Ph-OCH₃ 3.42-3.35 (m, H): Ph-CH₂—CH(P)-pyridine 3.29-3.2 and 3.05-2.97 (2m, 1H each): Ph-CH₂—CH(P)-pyridine 2.50 (s, 3H): Py-CH₃ 2.12 (1s, 3H): Ph-CH₃ 1.30 and 1.15 (2t, J=7 Hz, 3H each): P—O—CH₂—CH₃

### Example 5

### (E)-Diethyl β-(3-ethoxy-4-hydroxyphenyl)-α-(3-pyridyl)-vinyl Phosphonate

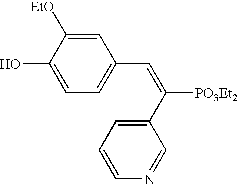

4-t-Butyldimethylsilyloxy-3-ethoxybenzaldehyde (2.69 g, 9.61 mmol) was prepared by reacting 3-ethoxy-4-hydroxybenzaldehyde (1.62 g, 9.7 mmol) with t-butyldimethylsilyl chloride (2.20 g, 14.6 mmol) in 40 ml DMF in presence of imidazole (2.19 g, 32.2 mmol). The whole procedure was carried out at −78° C. and under a nitrogen atmosphere. Diisopropylamine (3.4 ml, 24 mmol) was added dropwise to a solution of nBuLi 1.6 M (15 ml, 24 mmol) in 100 ml TBF. After 30 min. a solution of diethyl 3-pyridylmethylphosphonate (2.20 g, 9.61 mmol) in 10 ml TBF was added dropwise. After 30 min of stirring trimethyl chlorosilane (1.82 ml, 14.4 mmol) was added dropwise, the reaction mixture was stirred for a further 30 min then a solution of 4-t-butyldimethylsilyloxy-3-ethoxybenzaldehyde (2.69 g, 9.61 mmol) in 10 ml THF was added dropwise. The reaction mixture was stirred at −78° for 2 h, then the cooling bath was removed and saturated NH₄Cl-solution (100 ml) was added in one portion. The mixture was allowed to warm to room temperature and the aqueous phase was separated and extracted with ether. The combined organic layers were dried with MgSO₄ and evaporated to give 6.0 g of a brown oil. Purification of this crude product by column chromatography (CH₂Cl₂/MeOH 95/5) yielded 1.7 g (3.46 mmol, 36%) of (E)-diethyl β-(4-t-butyldimethylsilyloxy-3-ethoxyphenyl)-α-(3-pyridyl)-vinylphosphonate as a brown oil.

A solution of tetrabutylammonium fluoride (2.25 g, 7.13 mmol) in 30 ml TBF was added in four portions to a solution of the preceding compound (1.7 g, 3.46 mmol) in 20 ml TBF and 1.2 ml acetic acid. The reaction solution was stirred at room temperature for 3 h and was partitioned between CH₂Cl₂ and H₂O. The organic phase was separated and washed with three portions of saturated NaHCO₃ solution. The organic layer was dried with MgSO₄ and evaporated to give 1.8 g of a brown oil. Purification of this crude product by column chromatography (CH₂Cl₂/MeOH 95/5) yielded 0.51 g (1.35 mmol, 39%) of the title compound as an oil which slowly crystallized.

MS (m/e)=377: M⁺, 239 (100%): M⁺—HPO₃Et₂ NMR (CDCl₃): δ=8.59, 8.51, 7.65 and 7.35 (4m, H each): aromatic H, 3-pyridyl 7.62: (d, 1H, J=24 Hz): (Ph)(CH)C═C(P)-pyridine 6.77, 6.70 and 6.4 (3m, 1H each): aromatic H, substituted phenyl 6.15 (broad peak, 1H): OH 4.16-4.06 (m, 4H): P—O—CH₂—CH₃ 3.67 (q, J=7 Hz, 4H): PhO-CH₂—CH₃ 1.29 (2t, J=7 Hz, 6H): P—O—CH₂—CH₃ 1.27 (t, J=7 Hz, 3H): PhO-CH₂—CH₃

### Example 6

### (E)-Diethyl β-(3-ethoxy-4-hydroxyphenyl)-α-(3-pyridyl)-ethylphosphonate

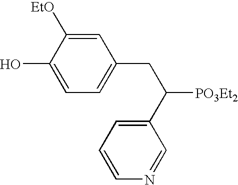

A solution of (E)-diethyl β-(3-ethoxy-4-hydroxyphenyl)-α-(3-pyridyl)vinylphosphonate (0.51 g, 1.38 mmol) in 80 ml ethanol was hydrogenated over 0.2 g of 10% Pd/C catalyst in a Parr hydrogenation apparatus at an initial pressure of 50 psi. When hydrogen uptake has ceased, the catalyst was filtered off, the solvent was evaporated to give 0.49 g (95%) of a yellow oil which slowly solidified.

MS (m/e)=379: M⁺, 241 (100%): M⁺—HPO₃Et₂ NMR (CDCl₃): δ=8.45, 8.37, 7.68 and 7.22 (4m, 1H each): aromatic H, 3-pyridyl 6.70, 6.48 and 6.37 (3m, 1H each): aromatic H, substituted phenyl 5.65 (s, 1H): OH 4.15-3.82 (3m, 4H total): P—O—CH₂—CH₃ and PhO-CH₂—CH₃ 3.47-3.39 (m, 1H): Ph-CH₂—CH(P)-pyridine 3.3-3.2 and 3.09-3.0 (2m, 1H each): Ph-CH₂—CH(P)-pyridine 1.34 and 1.15 (2t, J=7 Hz, 3H each): P—O—CH₂—CH₃ 1.30 (t, 3H): PhO-CH₂—CH₃

### Example 7

### Diethyl β-(4-hydroxy-3-methoxy-5-methylphenyl)-α-(2-pyrazinyl)-ethylphosphonate

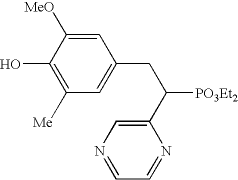

2-Chloromethylpyrazine was prepared by chlorination of 2-methylpyrazine by N-chlorosuccinimide in presence of dibenzoylperoxide in CCl₄ according to a literature method. The crude compound thus obtained was used directly for the next step. 60% NaH (4.36 g, 109 mmol) was washed three times with hexane and was suspended in 27 ml THF. This suspension was cooled to 0° and diethyl phosphite (14 ml, 109 mmol) was added dropwise. 30 Minutes after the end of the addition a solution of 2-chloromethylpyrazine (9.67 g, 75 mmol) in 40 ml THF was added dropwise and the ice bath was removed. The reaction was stirred for 4h then H₂O (20 ml) was added dropwise then a saturated NH₄Cl solution (20 ml) was added in one portion. The aqueous phase was separated and extracted with CHCl₃ (two 200 ml portions). The combined organic layers were dried with MgSO₄ and evaporated to give 16.8 g of a brown oil. Purification of this crude product by flash chromatography (CH₂Cl₂/MeOH 49:1, then 19:1) yielded 5.65 g (24.5 mmol, 33%) of diethyl 2-pyrazinylmethylphosphonate as a brown oil.

Diisopropyl amine (5.1 ml, 36 mmol) was added dropwise to a solution of nBuLi 1.6 M (22.5 ml, 36 mmol) in 130 ml TBF. After 30 min. a solution of diethyl 2-pyrazinylmethylphosphonate (2.75 g, 11.9 mmol) in 7 ml THF was added dropwise (int. temp.≦−70°). After 0.5 h TMSCl (3.0 ml, 23.7 mmol) was added dropwise (int. temp.≦−70°), further 30 min. later a solution of 4-t-butyldimethylsilyloxy-3-methoxy-5-methylbenzaldehyde (3.35 g, 11.9 mmol) in 9 ml THF was added dropwise (int. temp.≦−70°). The reaction mixture was stirred at −78° for 2 h, then the cooling bath was removed and a saturated NH₄Cl solution (50 ml) was added in one portion. The mixture was allowed to warm to room temperature and the aqueous phase was separated and extracted with ether (one 800 ml and three 300 ml portions). The combined organic layers were dried with MgSO₄ and evaporated to give 6.36 g of a brown oil. Purification of this crude product by flash chromatography (AcOEt, then AcOEt/MeOH 19:1) yielded 2.0 g (4.06 mmol, 34%) of diethyl β-(4-t-butyldimethylsilyloxy-3-methoxy-5-methylphenyl)-α-(2-pyrazinyl)-vinylphosphonate as a brown oil.

A solution of tetrabutylammonium fluoride (320 mg, 1.01 mmol) in 27 ml TBF was added in one portion to a solution of the preceding compound (2.00 g, 4.06 mmol) in 27 ml TBF. The reaction solution was stirred at room temperature for 3 h and was partitioned between 240 ml CH₂Cl₂ and 18 ml H₂O. The organic phase was separated and washed with 300 ml saturated NaHCO₃ solution. The organic layer was dried with MgSO₄ and evaporated to give 1.70 g of a brown oil. Purification of this crude product by flash chromatography (AcOEt, then AcOEt/MeOH 49:1) yielded 1.05 g (2.78 mmol, 68%) diethyl β-(4-hydroxy-3-methoxy-5-methylphenyl)-α-(2-pyrazinyl)-vinylphosphonate as a light brown oil.

MS (m/e): 378: M⁺, 241: M⁺—PO₃Et₂ ¹H-NMR (CDCl₃): δ=8.68 (t, 3=1.9 Hz, 1H): aromatic H, pyrazinyl 8.52 (m, 2H): aromatic H, pyrazinyl 7.78 and 7.73 (2s, 1H total): olefinic H (cis+trans) 6.49 and 6.17 (2d, J=1.3 Hz and J=1.8 Hz, 2H total): aromatic H, subst. phenyl 5.88 (s, 1H): OH 4.21-4.08 (m, 4H): P—O—CH₂—CH₃ 3.54 (s, 3H): Ph-OCH₃ 2.10 (s, 3H): Ph-CH₃ 1.33 and 1.28 (m, 6H total): P—O—CH₂—CH₃

A solution of the preceding compound (830 mg, 2.19 mmol) in 150 ml n-propanol was added in one portion to a solution of NaBH₃CN (1.65 g, 26.3 mmol) and ZnCl₂ (1.79 g, 13.1 mmol) in 50 ml MeOH. The reaction solution was heated to reflux and methanol was gradually evaporated until the boiling point of the remaining suspension reached 85°. The oil bath temperature was reduced to 90° and stirring was continued for 21 h. The reaction mixture was concentrated to about half its volume and the remaining suspension was partitioned between CHCl₃ and 10% NaOH. The aqueous phase was separated and extracted with CHCl₃. The combined organic layers were washed with H₂O, dried with MgSO₄ and evaporated to give 740 mg of a brown oil. Purification of this crude product by flash chromatography (AcOEt/MeOH 49:1, then 19:1) yielded 464 mg (1.22 mmol, 56%) of the title compound as an oil which slowly crystallized.

MS (m/e): 380: M⁺, 243: M⁺—PO₃Et₂ ¹H-NMR (CDCl₃): δ=8.53 (m, 1H): aromatic H, pyrazinyl 8.45 (t, J=1.6 Hz, 1H): aromatic H, pyrazinyl 8.39 (t, 3=2.3 Hz, 1H): aromatic H, pyrazinyl 6.42 and 6.33 (2d, J=1.4 Hz and 1.8 Hz, 2H total): aromatic H, substituted phenyl 5.53 (s, 1H): OH 4.19-4.00 (m, 4H): P—O—CH₂—CH₃ 3.71 (s, 3H): Ph-OCH₃ 3.68-3.60 (m, 1H): (Ph)CH₂—CH(P) 3.44-3.31 (m, 2H): (Ph)CH₂—CH(P) 2.11 (s, 3H): Ph-CH₃ 1.30 and 1.24 (2 t, J=7.1 Hz, 6H total): P—O—CH₂—CH₃

### Example 8

### (E)- and (Z)-Diethyl β-(3-methoxyphenyl)-α-(3-pyridyl)-vinyl Phosphonate

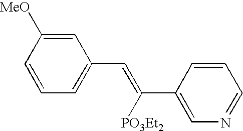

### (Z)-Diethyl β-(3-methoxyphenyl)-α-3-pyridyl)-vinyl Phosphonate

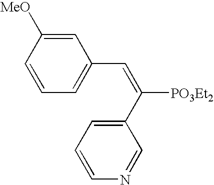

(E)-Diethyl β-(3-methoxyphenyl)-α-(3-pyridyl)-vinyl phosphonate diisopropylamine (7.72 ml, 54.6 mmol) was added dropwise to a solution of nBuLi 1.6 M (34.1 ml, 54.6 mmol) in 150 ml THF. After 30 min. a solution of diethyl 3-pyridylmethylphosphonate (5.0 g, 21.83 mmol) in 10 ml THF was added dropwise (int. temp.≦−70°). After 0.5h TMSCl (4.13 ml, 32.75 mmol) was added dropwise (int. temp.≦−70°), further 30 min. later a solution of 3-methoxybenzaldehyde (3.56 g, 26.2 mmol) was added dropwise (int. temp.≦−70°). The reaction mixture was stirred at −78° for 2 h, then the cooling bath was removed and sat. NH₄Cl-solution was added. The mixture was allowed to come to room temperature and the aqueous phase was separated and extracted with ether. The combined organic layers were dried with MgSO₄ and evaporated to give 8.5 g of a brown oil. Purification of this crude product by flash chromatography (CHCl₃/MeOH 95:5) yielded 2.08 g (6 mmol, 28%) of (E)-diethyl β-(3-methoxyphenyl)-α-(3-pyridyl)-vinylphosphonate and 0.18 g (0.5 mmol, 2.4%) of (Z)-Diethyl β-(3-methoxyphenyl)-α-(3-pyridyl)-vinylphosphonate as yellow oils.

The two stereoisomers were identified according to the following spectroscopic data: (E)-diethyl β-(3-methoxyphenyl)-α-(3-pyridyl)-vinyl phosphonate MS (m/e)=347: M⁺, 346 (100%): M⁺−1,210: M⁺—PO₃Et₂ NMR (CDCl₃): δ=8.57, 8.47, 7.65 and 7.32 (4m, H each): aromatic H, 3-pyridyl 7.7: (d, 1H, J=24 Hz): (Ph)(CH)C═C(P)-pyridine 7.11, 6.78, 6.66 and 6.52 (4m, 1H each): aromatic H, substituted phenyl 4.18-4.08 (m, 4H): P—O—CH₂—CH₃ 3.55 (s, 6H): Ph-OCH₃ 1.30 (t, 6H): P—O—CH₂—CH₃

(Z)-diethyl β-(3-methoxyphenyl)-α-(3-pyridyl)-vinyl phosphonate MS (m/e)=347: M⁺, 346 (100%): M⁺−1,210: M⁺—PO₃Et₂ NMR (CDCl₃): δ 8.68, 8.58, 7.87 and 7.30 (4m, H each): aromatic H, 3-pyridyl 7.32: (d, 1H, J=45 Hz): (Ph)(CH)C═C(P)-pyridine 7.11, 6.78, 6.66 and 6.52 (4m, 1H each): aromatic H, substituted phenyl 3.98-3.80 (m, 4H): P—O—CH₂—CH₃ 3.87 (s, 6H): Ph-OCH₃ 1.06 (t, 6H): P—O—CH₂—CH₃

### Example 9

### Diethyl β-(3-methoxyphenyl)-α-(3-pyridyl)-ethylphosphonate

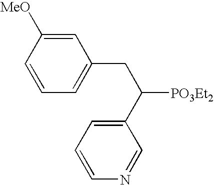

A solution of a mixture of (E)-and (Z)-diethyl β-(3-methoxyphenyl)-α-(3-pyridyl)-vinylphosphonate (1 g, 2.88 mmol) in 50 ml ethanol was hydrogenated over 0.5 g of 10% Pd/C catalyst in a Parr hydrogenation apparatus at an initial pressure of 50 psi. When hydrogen uptake has ceased, the catalyst was filtered off, the solvent was evaporated to give 0.78 g (2.23 mmol, 77%) of the title compound as a yellow oil.

MS (m/e)=349: M⁺, 211 (100%): M⁺—HBPO₃Et₂ NMR (CDCl₃): δ=8.44, 8.37, 7.72 and 7.22 (4m, 1H each): aromatic H, 3-pyridyl 7.07, 6.65, 6.57 and 6.51 (4m, 1H each): aromatic H, substituted phenyl 4.15-3.79 (3m, 4H total): P—O—CH₂—CH₃ 3.68 (s, 3H): Ph-OCH₃ 3.52-3.46 (m, H): Ph-CH₂—CH(P)-pyridine 3.36-3.28 and 3.17-3.07 (2m, 1H each): Ph-CH₂—CH(P)-pyridine 1.31 and 1.14 (2t, J=7 Hz, 3H each): P—O—CH₂—CH₃

### Example 10

### (Z)-Diethyl β-(3,4,5-trimethoxyphenyl)-α-(3-picolyl)-vinyl Phosphonate

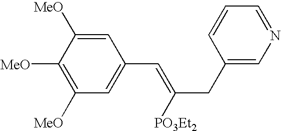

Diethyl 2-(3-pyridyl)ethylphosphonate was prepared according to the following procedure: 60% NaH (21.2 g, 53 mmol) was suspended in 250 ml THF. This suspension was cooled to 0° and tetraethyl methylenediphosphonate (72.63 ml, 28 mmol) was added dropwise. 30 Minutes after the end of the addition, pyridine-3-carboxaldehyde (28.53 g, 27 mmol) in 60 ml THF was added dropwise and the ice bath was removed. The mixture was stirred at room temperature for 4 h then H₂O (100 ml) was added dropwise followed by a saturated NH₄Cl solution (100 ml). The aqueous phase was separated and extracted with CHCl₃ (3 portions of 300 ml). The combined organic layers were dried with MgSO₄ and evaporated to give 44 g of a brown oil. Purification of this crude product by column chromatography (CH₂Cl₂/MeOH 9/1) yielded 38.5 g (17 mmol, 59%) of diethyl 2-(3-pyridyl)vinylphosphonate. A 50 ml ethanol solution of this compound (38 g, 16 mmol) was hydrogenated over 11 g of 10% Pd/C to give 36 g (148 mmol, 92%) of diethyl 3-pyridylethylphosphonate.

In the following step, the whole procedure was carried out at −78° C. and under a nitrogen atmosphere. N,N,N′,N′-tetramethylethylenediamine (7.4 ml, 49 mmol) was added dropwise to a solution of nBuLi 1.6 M (30.9 ml, 49 mmol) in 100 ml THF. After 30 min. a solution of diethyl 2-(3-pyridyl)ethylphosphonate (4 g, 16.5 mmol) in 7 ml THF was added dropwise. After 30 min of stirring trimethyl chlorosilane (4.2 ml, 33 mmol) was added dropwise, the reaction mixture was stirred for a further 30 min then a solution of 3,4,5-trimethoxybenzaldehyde (3.2 g, 17 mmol) in 15 ml THF was added dropwise. The reaction mixture was stirred at −78° for 2 h, then the cooling bath was removed and saturated NH₄Cl solution (70 ml) was added in one portion. The mixture was allowed to warm to room temperature and the aqueous phase was separated and extracted with ether. The combined organic layers were dried with MgSO₄ and evaporated to give 8 g of a brown oil. Purification of this crude product by flash chromatography (AcOEt/MeOH 9/1) yielded 2.01 g (4.8 mmol, 29%) of (Z)-diethyl β-(3,4,5-trimethoxyphenyl)-α-(3-picolyl)-vinylphosphonate as a yellow oil.

MS (m/e)=421 (100%): M⁺, 284: M⁺—PO₃Et₂ NMR (CDCl₃): δ=8.57, 8.50, 7.66 and 7.28 (4m, H each): aromatic H, 3-pyridyl 7.40: (d, J=47 Hz, 1H): (Ph)(CH)C═C(P)—CH₂-pyridine 6.87 (s, 2H): aromatic H, substituted phenyl 3.92-3.76 (m, 4H): P—O—CH₂—CH₃ 3.87 (s, 6H) and 3.85 (s, 3H): Ph-OCH₃ 3.78 (d, J=14 Hz, 2H): (Ph)(CH)C═C(P)—CH₂-pyridine 1.07 (t, 6H): P—O—CH₂—CH₂

### Example 11

### (E)-Diethyl β-(3,4,5-trimethoxyphenyl)-α-(3-picolyl)-vinylphosphonate

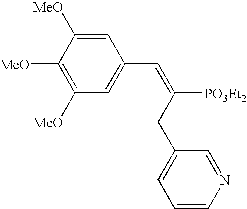

Tetraethyl 2-(3-pyridyl)ethylene-1,1-diphosphonate was prepared according to the following procedure: Under a nitrogen atmosphere, titanium tetrachloride (41 ml, 369 mmol) was added dropwise to a 600 ml of THF solution cooled to 0° C. by means of an ice bath, followed by pyridine-3-carboxaldehyde (18 g, 168 mmol). Tetraethyl methylenediphosphonate (53.3 g, 183 mmol) dissolved in 60 ml THF was added dropwise, followed by N-methylmorpholine (75 g, 741 mmol) and the resulting mixture was stirred at room temperature overnight. The reaction mixture was then partitioned between water and chloroform, the organic phase was washed until neutral pH, dried over MgSO4 and evaporated. The residue was purified by column chromatography (CHCl₃/MeOH 95/5) to give 11.5 g (30 mmol, 18%) of tetraethyl 2-(3-pyridyl)ethenylidene-1,1-diphosphonate a brown oil. A 100 ml ethanol solution of this compound (11.5 g, 30 mmol) was hydrogenated over 2 g of 10% Pd/C to give 2.73 g (7.2 mmol, 24%) of tetraethyl 2-(3-pyridyl)ethylidene-1,1-diphosphonate.

In the following step, the whole procedure was carried out at −78° C. and under a nitrogen atmosphere. Diisopropylamine (2.8 ml, 20 mmol) was added dropwise to a solution of nBuLi 1.6 M (12.4 ml, 20 mmol) in 100 ml THF. After 30 min. a solution of tetraethyl 2-(3-pyridyl)ethylidenediphosphonate (2.5 g, 6.6 mmol) in 7 ml TBF was added dropwise. After 30 min of stirring a solution of 3,4,5-trimethoxybenzaldehyde (1.3 g, 6.6 mmol) in 9 ml THF was added dropwise. The reaction mixture was stirred at −78° for 2 h, then the cooling bath was removed and saturated NH₄Cl solution (50 ml) was added in one portion. The mixture was allowed to warm to room temperature and the aqueous phase was separated and extracted with ether. The combined organic layers were dried with MgSO₄ and evaporated to give 3.5 g of a brown oil. Purification of this crude product by flash chromatography (AcOEt/MeOH 9/1) yielded 0.68 g (1.6 mmol, 24%) of (E)-diethyl β-(3,4,5-trimethoxyphenyl)-α-(3-picolyl)-vinylphosphonate as a yellow oil. The (Z)-isomer was isolated as a secondary product (0.35 g, 12%).

MS (m/e)=421 (100%): M⁺, 283: M⁺—HPO₃Et₂ NMR (CDCl₃): δ=8.53, 8.46, 7.58 and 7.22 (4m, H each): aromatic H, 3-pyridyl 7.71: (d, J=24 Hz, 1H): (Ph)(CH)C═C(P)CH₂-pyridine 6.55 (s, 2H): aromatic H, substituted phenyl 4.12-3.96 (m, 4H): P—O—CH₂—CH₃ 3.93 (d, J=19.5 Hz, 2H): (Ph)(CH)C═C(P)—CH₂-pyridine 3.84 (s, 3H) and 3.67 (s, 6H): Ph-OCH₃ 1.22 (t, 6H): P—O—CH₂—CH₃

### Example 12

### Diethyl β-(3,4,5-trimethoxyphenyl)-α-(3-picolyl)-ethylphosphonate

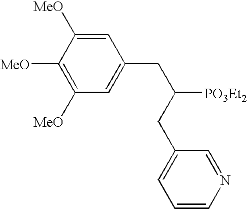

A solution of a mixture of (E)-and (Z)-diethyl β-(3,4,5-trimethoxyphenyl)-α-(3-picolyl)-vinylphosphonate (0.8 g, 1.9 mmol) in 50 ml ethanol was hydrogenated over 0.3 g of 10% Pd/C catalyst in a Parr hydrogenation apparatus at an initial pressure of 50 psi. When hydrogen uptake has ceased, the catalyst was filtered off, the solvent was evaporated to give 0.6 g of a dark oil. This crude product was purified by column chromatography (AcOEt/MeOH 9/1) to yield 0.41 g (0.97 mmol, 51%) of the title compound as a yellow oil.

MS (m/e)=423: M⁺, 285 (100%): M⁺—HPO₃Et₂ NMR (CDCl₃): δ=8.41, 8.38, 7.37 and 7.13 (4m, 1H each): aromatic H, 3-pyridyl 6.32 (s, 2H): aromatic H, substituted phenyl 4.08-3.90 (3m, 4H total): P—O—CH₂—CH₃ 3.81 (s, 9H): Ph-OCH₃ 3.22-3.14, 3.06-2.96, 2.81-2.71 and 2.64-2.55 (4m, 1H each): Ph-CH₂—CH(P)—CH₂-pyridine 2.45-2.34 (m, 1H): Ph-CH₂—CH(P)—CH₂-pyridine 1.25 and 1.20 (2t, J=7 Hz, 3H each): P—O—CH₂—CH₃

### Example 13

### Summary of Synthesized Compounds

Summarized in TABLE 1 are a number of α-substituted-heteroarylalkylphosphonates of formula (I) where X⁵ is H and n=0.

TABLE 1

| Cpd | X¹ | X² | X³ | X⁴ | m | Formula | Het | R¹, R² |
|---|---|---|---|---|---|---|---|---|
| 1 | H | OMe | H | H | 0 | (Ia^{Z}) | 3-pyridyl | Et |
| 2 | H | OMe | H | H | 0 | (Ia^{E}) | 3-pyridyl | Et |
| 3 | H | OMe | H | H | 0 | (Ib) | 3-pyridyl | Et |
| 4 | H | OMe | OMe | OMe | 0 | (Ia^{E}) | 3-pyridyl | Et |
| 5 | H | OMe | OMe | OMe | 0 | (Ia^{Z}) | 3-pyridyl | Et |
| 6 | H | OMe | OMe | OMe | 1 | (Ia^{Z}) | 3-pyridyl | Et |
| 7 | H | OMe | OMe | OMe | 1 | (Ia^{E}) | 3-pyridyl | Et |
| 8 | H | OMe | OMe | OMe | 1 | (Ib) | 3-pyridyl | Et |
| 9 | OMe | H | OMe | OMe | 0 | (Ia^{E}) | 3-pyridyl | Et |
| 10 | OMe | H | OMe | OMe | 0 | (Ib) | 3-pyridyl | Et |
| 11 | OMe | H | OMe | OMe | 1 | (Ia^{Z}) | 3-pyridyl | Et |
| 12 | OMe | H | OMe | OMe | 1 | (Ib) | 3-pyridyl | Et |
| 13 | H | OEt | OH | H | 0 | (Ia^{E}) | 3-pyridyl | Et |
| 14 | H | OEt | OH | H | 0 | (Ib) | 3-pyridyl | Et |
| 15 | Me | Me | OH | Me | 0 | (Ia^{E}) | 3-pyridyl | Et |
| 16 | Me | Me | OH | Me | 0 | (Ia^{E}) | 3-pyridyl | Et |
| 17 | H | OMe | OH | OMe | 0 | (Ia^{E}) | 3-pyridyl | Et |
| 18 | H | OMe | OH | OMe | 0 | (Ib) | 3-pyridyl | Et |
| 19 | H | OMe | OH | OMe | 0 | (Ia^{E}) | 5-(2-methyl | Et |
| | | | | | | | pyridyl) | |
| 20 | H | OMe | OH | OMe | 0 | (Ib) | 5-(2-methyl | Et |
| | | | | | | | pyridyl) | |
| 21 | H | OMe | OH | OMe | 0 | (Ib) | 5-(2-methyl | iPr |
| | | | | | | | pyridyl) | |
| 22 | H | OMe | OH | Me | 0 | (Ia^{E}) | 3-pyridyl | Et |
| 23 | H | OMe | OH | Me | 0 | (Ib) | 3-pyridyl | Et |
| 24 | H | OMe | OH | Me | 0 | (Ia^{E}) | 5-(2-methyl | Et |
| | | | | | | | pyridyl) | |
| 25 | H | OMe | OH | Me | 0 | (Ib) | 5-(2-methyl | Et |
| | | | | | | | pyridyl) | |
| 26 | H | OMe | OH | Me | 0 | (Ib) | 5-(2-methyl | iPr |
| | | | | | | | pyridyl) | |
| 27 | H | OMe | OH | OMe | 0 | (Ia^{E}) | 4-(2-methyl | Et |
| | | | | | | | thiazolyl) | |
| 28 | H | OMe | OH | Me | 0 | (Ia^{E}) | 4-(2-methyl | Et |
| | | | | | | | thiazolyl) | |
| 29 | H | OMe | OH | Me | 0 | | pyrazinyl | Et |
| 30 | H | OMe | OH | Me | 0 | (Ib) | pyrazinyl | Et |

### Example 14

### Biological Data

A. Lp(a) Lowering Activity

1. In Vitro Data

The compounds of formula (1) were assayed for being able to effectively lower the production of apo (a) in primary cultures of Cynomolgus hepatocytes.

Protocol—Hepatocytes were isolated from livers of male adult Cynomolgus monkeys by the two-step collagenase perfusion method according to C. Guguen-Guillouzo and A. Guillouzo “Methods for preparation of adult and fetal hepatocytes” p. 1-12 in “Isolated and Cultured Hepatocytes”, les editions Inserm Paris and John Libbey Eurotext London (1986).

The viability of cells was determined by Trypan blue staining. The cells were then seeded at a density of 1.5-2.10⁵ viable cells per 2 cm² in 24 well tissue culture plates in a volume of 500 μl per well of Williams E tissue culture medium containing 10% fetal calf serum. Cells were incubated for 6-24 hours at 37° C. in a CO₂ incubator (5% CO₂) in the presence of 20 μM of the test compounds dissolved in ethanol. Four wells were used for each compound. Nicotinic acid and steroid hormones were used as references to validate the assay system since they are known to decrease apo (a) in man. Control cells were incubated in the presence of ethanol only.

The amount of apo (a) secreted in culture medium was assayed directly by ELISA using a commercially available kit. Changes in apo (a) concentration in culture medium are given as the percentage of value measured for the control plates.

Results—The compounds No. 4, 5, 6, 7, 9, 10, 13, 14, 15, 16. 17, 18, 19, 20, 22, 23, 24 and 25 tested at 20 μM were found to lower the apo (a) secretion in the range between −15% to −40%.

2. In Vivo Data

Study Protocol—Male cynomolgus monkeys weighing between 3 and 7 kg were divided into groups of 3 to 4 animals each. Prior to treatment their plasma Lp(a) levels were followed over a two-month period to ascertain a constant baseline value. Test compounds were given orally by gavage at the dose of 50 mg/kg/day for 2 weeks and Lp(a) was measured at days 7 and 14. At the end of the dosing period, animals were maintained for a treatment free period of 4 weeks, whereupon the decreased plasma Lp(a) levels returned to pretreatment levels. This control provided proof that the decrease in Lp(a) measured was caused by the pharmacological activity of the test compounds. At Days −1 and 7 or 14, after an overnight fast blood samples were collected on EDTA and Lp(a) was measured by the highly sensitive and specific ELISA test. Results (mean of 34 values of each group) were expressed as % of pre-dose (Day −1).

Results—Selected compounds of formula (1) were tested under the experimental conditions to investigate their pharmacological activity in vivo. The compounds No 23 and 25 lower plasma Lp(a) in the range of −20% to −29% (values measured at Day 7 or 14, % changes from pre-dose at Day −1).

B. Cholesterol Lowering Activity

Study Protocol. Male cynomolgus monkeys weighing between 3 and 7 kg are divided into groups of 3 to 4 animals each. Prior to treatment, their plasma cholesterol, LDL cholesterol and apo B levels are followed over a one-month period to ascertain a constant baseline value. Test compounds are given orally by gavage at the dose of 50 mg/kg/day for 2 weeks and apo B, LDL cholesterol, and total plasma cholesterol are measured at days 7 and 14. At the end of the dosing period, animals are maintained for a treatment-free period of 4 weeks, whereupon their cholesterol levels returned to pre-treatment levels. This control provides proof that the decrease in cholesterol measured is caused by the pharmacological activity of the test compounds. At Days −1 and 7 or 14, after an overnight fast, blood samples are collected on EDTA and apo B is measured by an ELISA method (Morwell diagnostics), LDL cholesterol by an immuno turbidimetric method (Boehringer) and total plasma cholesterol by an enzymatic method (CHOD-PAP, Boehringer). Results (mean of 3-4 values of each group) are expressed as % of pre-dose (Day −1).

1. A compound of formula (Ia): 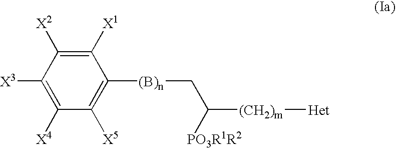
or a compound of formula (Ib): 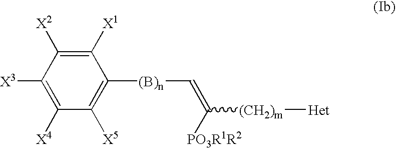
in which:
X¹, X², X³, X⁴ and X⁵ are independently hydrogen, hydroxy, hydroxymethyl, C₁-C₃ alkoxymethyl, straight or branched C₁-C₈ alkyl, straight or branched C₁-C₈ alkoxy, C₃-C₆ cycloalkyl, C₃-C₆ cycloalkoxy, cyano, nitro or halogen, wherein said halogen is fluoro, chloro, bromo or iodo; or
X² may be combined with X³, or X⁴ may be combined with X⁵, to form a 5- to 6-membered alkylidenedioxy ring optionally substituted with a C₁-C₄ alkyl group; or
X⁴ may be combined with X⁵ to form a 5- to 6-membered alkylidene ring optionally substituted with a C₁-C₄ alkyl group;
R¹ and R² are independently hydrogen or a straight or branched C₁-C₆ alkyl;
B is CH₂, CH₂—CH₂ or CH═CH;
n is zero or 1;
m is zero, 1 or 2;
Het is an optionally substituted heteroaryl group comprising at least one nitrogen atom, or a pharmaceutically acceptable salt thereof.

2. The compound of claim 1, wherein said compound is a compound of formula (Ia).

3. The compound of claim 1, wherein said compound is a compound of formula (Ib).

4. The compound of claim 3, wherein said compound of formula (Ib) is the Z-isomer, the E-isomer, or a mixture thereof.

5. The compound of claim 1, wherein X¹ is hydrogen, or methyl, X² is methoxy, ethoxy, methyl, tert-butyl or hydroxy, X³ is hydrogen, hydroxy, methoxy, methyl, ethyl or hydroxymethyl, X⁴ is hydrogen, methoxy, tert-butyl or methyl and X⁵ is hydrogen.

6. The compound of claim 5, wherein X² is methoxy, X³ is hydroxy and X⁴ is methyl or methoxy.

7. The compound of claim 5, wherein m is zero or 1.

8. The compound of claim 5, wherein n is zero.

9. The compound of claim 5, wherein R¹ and R² are independently C₁-C₃ alkyl.

10. The compound of claim 9, wherein R¹ and R² are independently ethyl or isopropyl.

11. The compound of claim 8, wherein m is zero or 1.

12. The compound of claim 1, wherein Het is optionally substituted and comprises a hetroaryl group selected from the group consisting of pyridyl, pyrimidyl, pyrazinyl, pyridazinyl, thiazolyl, thiadiazolyl, benzothiazolyl, pyrazolyl and triazinyl.

13. The compound of claim 12, wherein said heteroaryl group selected from the group consisting of pyridyl, pyrimidyl, pyrazinyl, pyridazinyl, thiazolyl, benzothiazolyl, pyrazolyl and triazinyl is substituted with one or two methyl groups or one or two methoxy groups or wherein said heteroaryl group is thiadiazolyl and is substituted by a methyl or a methoxy group.

14. The compound of claim 12, wherein Het is pyrazinyl, 3-pyridyl, 5-(2-methylpyridyl), 5-(2-methylthiazolyl) pyridyl)

15. The compound of claim 1, wherein said compound is selected from the group consisting of:
(E)-diethyl β-(3-ethoxy-4-hydroxyphenyl)-α-(3-pyridyl)vinylphosphonate;
diethyl β-(3-ethoxy-4-hydroxyphenyl)-α-(3-pyridyl)ethylphosphonate;
(E)-diethyl β-(4-hydroxy-2,3,5-trimethylphenyl)-α-(3-pyridyl)vinylphosphonate;
diethyl β-(4-hydroxy-2,3,5-trimethylphenyl)-α-(3-pyridyl)ethylphosphonate;
(E)-diethyl β-(3,5-dimethoxy-4-hydroxyphenyl)-α-(3-pyridyl)vinylphosphonate;
diethyl β-(3,5-dimethoxy-4-hydroxyphenyl)-α-(3-pyridyl)ethylphosphonate;
(E)-diethyl β-(3,5-dimethoxy-4-hydroxyphenyl)-α-(5-(2-methylpyridyl))vinylphosphonate;
diethyl β-(3,5-dimethoxy-4-hydroxyphenyl)-α-(5-(2-methylpyridyl))ethylphosphonate;
diisopropyl β-(3,5-dimethoxy-4-hydroxyphenyl)-α-(5-(2-methylpyridyl))ethylphosphonate;
(E)-diethyl β-(4-hydroxy-3-methoxy-5-methylphenyl)-α-(3-pyridyl)vinylphosphonate;
diethyl β-(4-hydroxy-3-methoxy-5-methylphenyl)-α-(3-pyridyl)ethylphosphonate;
(E)-diethyl β-(4-hydroxy-3-methoxy-5-methylphenyl)-α-(5-(2-methylpyridyl)) vinylphosphonate;
diethyl β-(4-hydroxy-3-methoxy-5-methylphenyl)-α-(5-(2-methylpyridyl)) ethylphosphonate;
diisopropyl β-(4-hydroxy-3-methoxy-5-methylphenyl)-α-(5-(2-methylpyridyl)) ethylphosphonate;
(E)-diethyl β-(3,5-dimethoxy-4-hydroxyphenyl)-α-(4-(2-methylthiazolyl))vinylphosphonate;
(E)-diethyl β-(4-hydroxy-3-methoxy-5-methylphenyl)-α-(4-(2-methylthiazolyl)) vinylphosphonate;
(E)-diethyl β-(4-hydroxy-3-methoxy-5-methylphenyl)-α-(pyrazinyl)vinylphosphonate; and
diethyl β-(4-hydroxy-3-methoxy-5-methylphenyl)-α-(pyrazinyl)ethylphosphonate.

16. A pharmaceutical composition comprising a compound of claimed in claim 1 and a pharmaceutically acceptable carrier.

17. A method for decreasing plasma levels of apo (a), lipoprotein(a), apo B, LDL cholesterol and total cholesterol, comprising administration to a patient in need of such treatment of an effective amount of a compound of claim 1.

18. A method for the treatment and/or prevention of thrombosis, comprising administration to a patient in need of such treatment an effective amount of a compound of claim 1.

19. A method for the treatment and/or prevention of restenosis following angioplasty, comprising administration of an amount effective to decrease plasma levels of apo (a) and lipoprotein(a) of a compound of claim 1.

20. A method for the treatment and/or prevention of atherosclerosis, comprising administration to a patient in need of such treatment an effective amount of a compound claim 1.

21. The method of claim 20, further comprising administering an effective amount of a cholesterol synthesis inhibitor.

22. The method of claim 21, wherein said cholesterol synthesis inhibitor is a statin selected from the group consisting of atorvastatin, simvastatin, pravastatin, cerivastatin, fluvastatin, lovastatin and ZD 4522.
